(19) United States
(12) Patent Application Publication
(10) Pub. No.: US 2017/0072197 A1
(43) Pub. Date: Mar. 16, 2017

(54) COCHLEAR IMPLANT HEARING ASSISTANCE SYSTEM AND METHOD OF FITTING THE SAME

(71) Applicant: ADVANCED BIONICS AG, Staefa (CH)

(72) Inventors: Daniel Alfsmann, Bochum (DE); Leonid M. Litvak, Los Angeles, CA (US)

(21) Appl. No.: 15/309,174

(22) PCT Filed: May 7, 2014
(86) PCT No.: PCT/US14/37200

§ 371 Date: Nov. 4, 2016

(51) Int. Cl. A61N 1/36; A61N 1/372; H04R 25/00; A61N 1/05

(52) U.S. Cl. CPC A61N 1/36032; A61N 1/0541; A61N 1/37247; H04R 25/70; H04R 25/353

(57) ABSTRACT

There is provided a hearing assistance system comprising a cochlear implant device for neural stimulation of the cochlea of one of the patient's ears and a fitting device for adjusting the cochlear implant device.

The invention relates to a hearing assistance system comprising a cochlear implant device for neural stimulation of the cochlea and a fitting device for adjusting the cochlear implant device.

Typically, cochlear implants comprise an electrode array for electrical stimulation of the cochlear at various stimulation sites determined by the position of the respective electrode. Systems for bimodal stimulation of the hearing comprise a cochlear implant at the ipsilateral ear and a device for acoustic stimulation of the ipsilateral ear or the contralateral ear. Systems with electric and acoustic stimulation of the same ear are also known as hybrid devices or EAS devices. In systems with contralateral acoustic stimulation the acoustic stimulation device typically is an (electro-acoustic) hearing aid.

In a cochlear implant (CI) a frequency allocation map specifies which frequency sub-ranges (frequency band) of the input audio signal (i.e. the audio signal provided by the microphone and/or an external audio source) are assigned to each stimulation channel, with the stimulation channels being formed by the implant electrodes. Over time, a CI patient adapts to his specific frequency allocation, so that a modification of the specific frequency allocation may result in an acceptance problem. For example, a need to modify the frequency allocation of the CI may occur in cases in which a patient first is provided with a CI with electrical stimulation only and later is provided in addition with acoustic stimulation of the same ear (EAS system) or of the other ear (bimodal system). Typically, the lower input signal frequencies are selected for acoustic stimulation, while the remaining higher input signal frequencies are selected for electrical stimulation.

U.S. Pat. No. 8,571,674 B2 relates to an iterative fitting method for a multimodal hearing assistant system including electrical and acoustic stimulation.

EP 1 702 496 B1 relates to fitting method for a CI device, wherein a fitting curve resulting from an allocation of the input frequencies to the output channels according to a logarithmic function can be manually adjusted with regard to its position and slope via a graphical user interface comprising a first slider for adjusting the slope and a second slider for adjusting the frequency axis intercept.

US 2005/0261748 A1 relates to a fitting method for a hybrid device used by a patient having residual acoustic hearing capability at the ipsilateral ear, wherein the portion of the cochlea having residual acoustic hearing capability is determined by measuring the neural response to acoustic and/or electrical stimulation.

US 2011/0238176 A1 likewise relates to a fitting method for a hybrid device, wherein a tonotopic response for the residual hearing of the ipsilateral cochlear is measured to obtain a place-frequency map.

U.S. Pat. No. 8,155,747 B2 relates to a method of fitting a bilateral hearing system comprising a CI device at one ear and a hearing aid at the other ear.

It is an object of the invention to provide for a hearing assistance system comprising a CI device and a fitting device, which allows for a frequency allocation in a manner which is both convenient to the user of the fitting device and acceptable to the CI patient. It is a further object to provide for a corresponding fitting method.

These objects are achieved by a system as defined in claim 1 and a method as defined in claim 22.

The invention is beneficial in that, by enabling the user to select a value of a matching frequency located in between the lower cutoff frequency and the upper cutoff frequency of the input audio signal, the input audio signals frequencies can be divided into two regions, namely a first region extending from the matching frequency to the upper cutoff frequency, which keeps its frequency allocation, and a second region extending from the modified lower cutoff frequency to the mapping frequency, wherein the frequency allocation can be modified in order to adapt it to the increased lower cutoff frequency. Thereby, the user of the fitting device is enabled to provide, in a very simple manner requiring selection of only a single parameter, namely the mapping frequency, for a relatively smooth transition between a previous frequency allocation and a modified frequency allocation having an increased lower cutoff frequency. This is particularly useful, for example, in case that a CI patient is later provided with additional acoustic stimulation which may require an increase of the lower cutoff frequency of the frequency range of the input audio signal supplied to electric stimulation.

Preferred embodiments of the invention are defined in the dependent claims.

Hereinafter, examples of the invention will be illustrated by reference to the attached drawings, wherein:

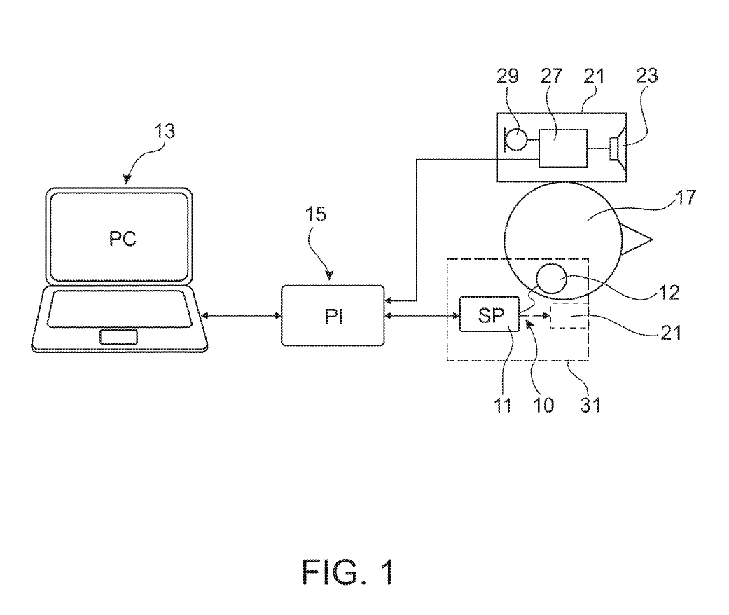

FIG. 1 is a schematic representation of an example of a system according to the invention;

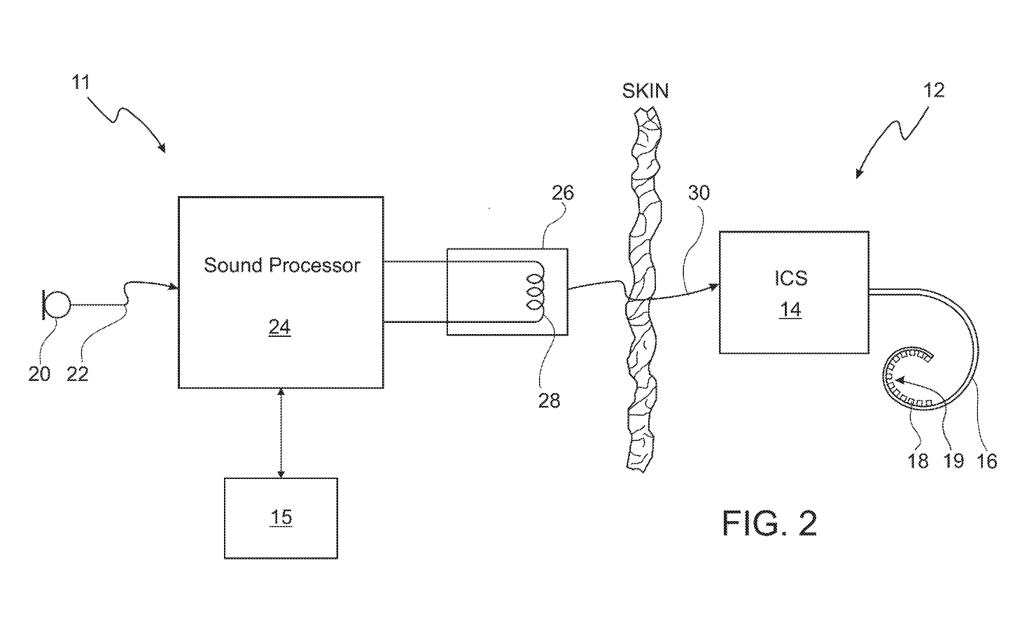

FIG. 2 is a schematic representation of an example of the CI device of FIG. 1;

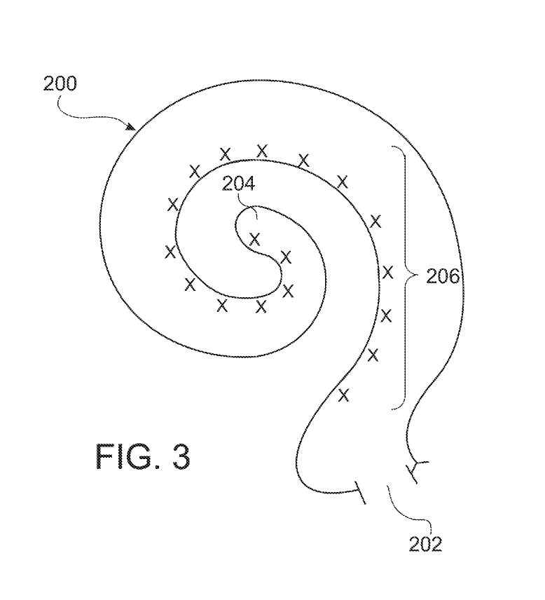

FIG. 3 is a schematic cross-sectional view of a human cochlea with marked stimulation sites;

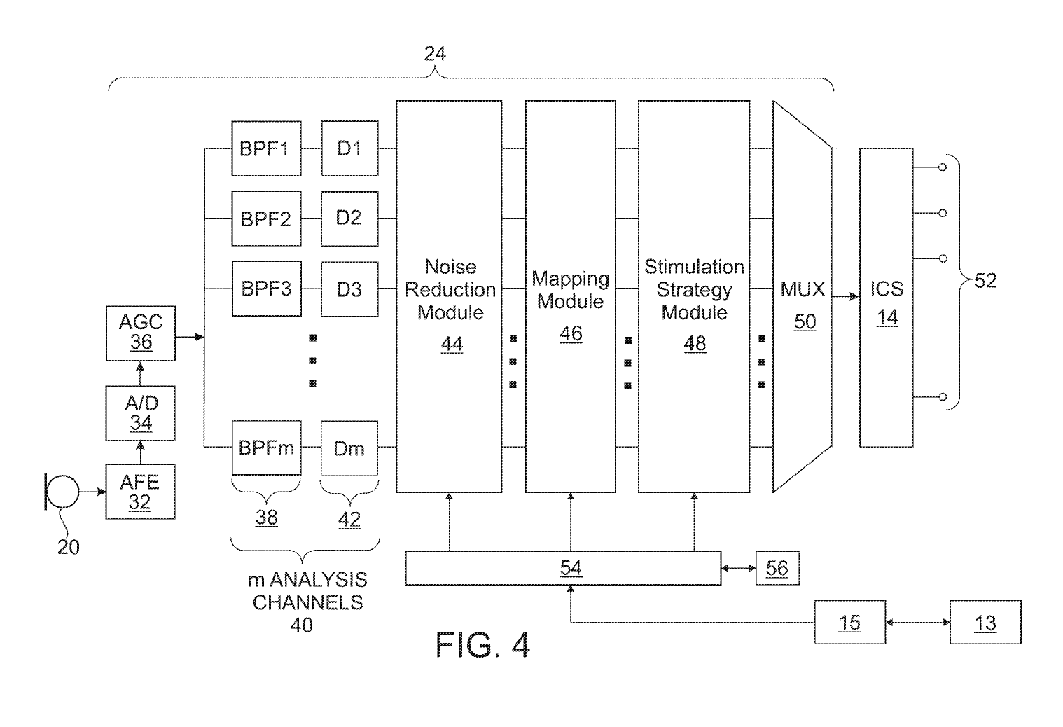

FIG. 4 is a block diagram of an example of the signal processing structure of a CI device to be used with the invention;

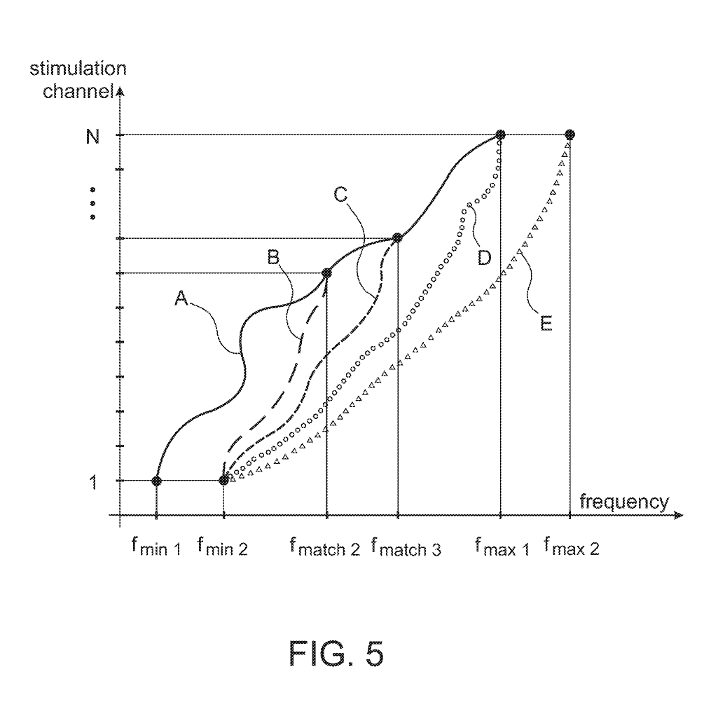

FIG. 5 is a schematic representation of several examples of the allocation of the input audio signal frequencies to the stimulation channels;

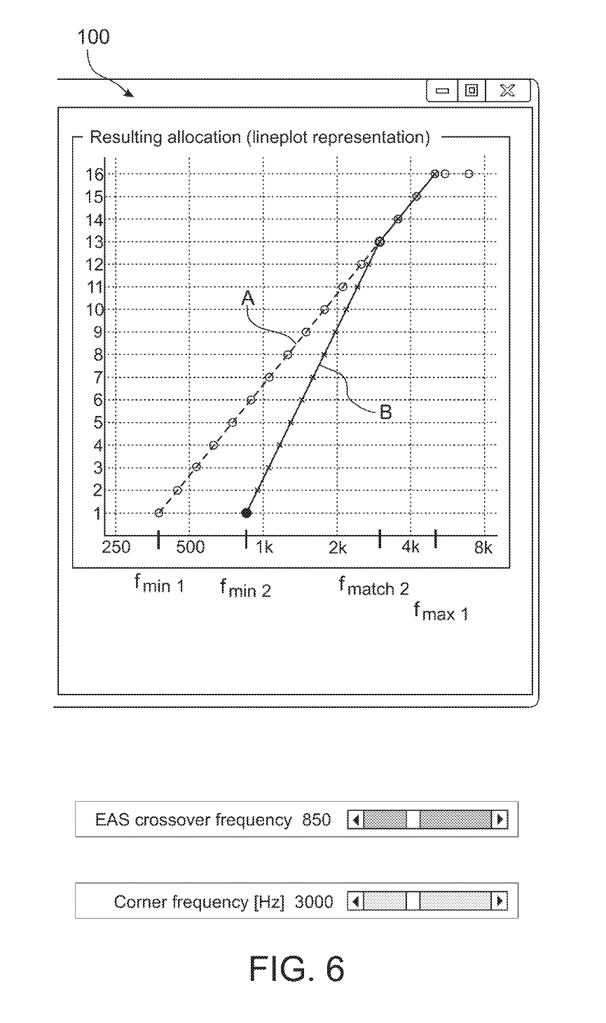

FIGS. 6-8 are representations of a graphical user interface of an example of a fitting device according to the invention, wherein different examples of logarithmic frequency allocations of an EAS system are shown.

FIG. 1 is a schematic representation of an example of a stimulation system according to the invention, comprising a fitting/programming unit 13, which may be implemented as a computer, a programming interface 15, and a CI device 10 comprising a sound processing subsystem 11 and an implantable stimulation subsystem 12 and being worn by a patient 17 at the ipsilateral ear. The programming unit 13 communicates with the sound processing subsystem 11 via the programming interface 15, which may be implemented as a wired or wireless connection.

It is to be understood that the programming unit 13 is used with the CI device 10 only for adjustment/fitting, but not during normal operation of the CI device 10.

In FIG. 2 an example of the cochlear implant device 10 of the system of FIG. 1 is shown schematically. The sound processing sub-system 11 serves to detect or sense an audio signal and divide the audio signal into a plurality of analysis channels, each containing a frequency domain signal (or simply “signal”) representative of a distinct frequency portion of the audio signal. A signal level value and optionally a noise level value are determined for each analysis channel by analyzing the respective frequency domain signal, and a noise reduction gain parameter may be determined for each analysis channel as a function of the signal level value and the noise level value of the respective analysis channel. Noise reduction may be applied to the frequency domain signal according to the noise reduction gain parameters to generate a noise reduced frequency domain signal. Stimulation parameters are generated based on the noise reduced frequency domain signal and are transmitted to the stimulation sub-system 12.

Stimulation sub-system 12 serves to generate and apply electrical stimulation (also referred to herein as “stimulation current” and/or “stimulation pulses”) to stimulation sites at the auditory nerve within the cochlea of a patient 17 in accordance with the stimulation parameters received from the sound processing sub-system 11. Electrical stimulation is provided to the patient 17 via a CI stimulation assembly 18 comprising a plurality of stimulation channels, wherein various known stimulation strategies, such as current steering stimulation or N-of-M stimulation, may be utilized.

As used herein, a “current steering stimulation strategy” is one in which weighted stimulation current is applied concurrently to two or more electrodes by an implantable cochlear stimulator in order to stimulate a stimulation site located in between areas associated with the two or more electrodes and thereby create a perception of a frequency in between the frequencies associated with the two or more electrodes, compensate for one or more disabled electrodes, and/or generate a target pitch that is outside a range of pitches associated with an array of electrodes.

As used herein, an “N-of-M stimulation strategy” is one in which stimulation current is only applied to N of M total stimulation channels during a particular stimulation frame, where N is less than M. An N-of-M stimulation strategy may be used to prevent irrelevant information contained within an audio signal from being presented to a CI user, achieve higher stimulation rates, minimize electrode interaction, and/or for any other reason as may serve a particular application.

The stimulation parameters may control various parameters of the electrical stimulation applied to a stimulation site including, but not limited to, frequency, pulse width, amplitude, waveform (e.g., square or sinusoidal), electrode polarity (i.e., anode-cathode assignment), location (i.e., which electrode pair or electrode group receives the stimulation current), burst pattern (e.g., burst on time and burst off time), duty cycle or burst repeat interval, spectral tilt, ramp-on time, and ramp-off time of the stimulation current that is applied to the stimulation site.

FIG. 3 illustrates a schematic structure of the human cochlea 200. As shown in FIG. 3, the cochlea 200 is in the shape of a spiral beginning at a base 202 and ending at an apex 204. Within the cochlea 200 resides auditory nerve tissue 206 which is organized within the cochlea 200 in a tonotopic manner. Low frequencies are encoded at the apex 204 of the cochlea 200 while high frequencies are encoded at the base 202. Hence, each location along the length of the cochlea 200 corresponds to a different perceived frequency. Stimulation subsystem 12 is configured to apply stimulation to different locations within the cochlea 200 (e.g., different locations along the auditory nerve tissue 206) to provide a sensation of hearing.

Returning to FIG. 2, sound processing subsystem 11 and stimulation subsystem 12 is configured to operate in accordance with one or more control parameters. These control parameters may be configured to specify one or more stimulation parameters, operating parameters, and/or any other parameter as may serve a particular application. Exemplary control parameters include, but are not limited to, most comfortable current levels (“M levels”), threshold current levels (“T levels”), dynamic range parameters, channel acoustic gain parameters, front and backend dynamic range parameters, current steering parameters, amplitude values, pulse rate values, pulse width values, polarity values, filter characteristics, and/or any other control parameter as may serve a particular application. In particular, the control parameters may include a frequency allocation table (FAT) which determines the respective frequency range allocated to a certain electrode.

In the example shown in FIG. 2, the stimulation sub-system 12 comprises an implantable cochlear stimulator (ICS) 14, a lead 16 and the stimulation assembly 18 disposed on the lead 16. The stimulation assembly 18 comprises a plurality of “stimulation contacts” 19 for electrical stimulation of the auditory nerve. The lead 16 may be inserted within a duct of the cochlea in such a manner that the stimulation contacts 19 are in communication with one or more stimulation sites within the cochlea, i.e. the stimulation contacts 19 are adjacent to, in the general vicinity of, in close proximity to, directly next to, or directly on the respective stimulation site.

In the example shown in FIG. 2, the sound processing sub-system 11 is designed as being located external to the patient 17; however, in alternative examples, at least one of the components of the sub-system 11 may be implantable.

In the example shown in FIG. 2, the sound processing sub-system 11 comprises a microphone 20 which captures audio signals from ambient sound, a microphone link 22, a sound processor 24 which receives audio signals from the microphone 20 via the link 22, and a headpiece 26 having a coil 28 disposed therein. The sound processor 24 is configured to process the captured audio signals in accordance with a selected sound processing strategy to generate appropriate stimulation parameters for controlling the ICS 14 and may include, or be implemented within, a behind-the-ear (BTE) unit or a portable speech processor (PSP). In the example of FIG. 2 the sound processor 24 is configured to transcutaneously transmit data (in particular data representative of one or more stimulation parameters) to the ICS 14 via a wireless transcutaneous communication link 30. The headpiece 26 may be affixed to the patient's head and positioned such that the coil 28 is communicatively coupled to the corresponding coil (not shown) included within the ICS 14 in order to establish the link 30. The link 30 may include a bidirectional communication link and/or one or more dedicated unidirectional communication links. According to an alternative embodiment, the sound processor 24 and the ICS 14 may be directly connected by wires.

In FIG. 4 a schematic example of a sound processor 24 is shown. The audio signals captured by the microphone 20 are amplified in an audio front end circuitry 32, with the amplified audio signal being converted to a digital signal by an analog-to-digital converter 34. The resulting digital signal is then subjected to automatic gain control using a suitable automatic gain control (AGC) unit 36.

After appropriate automatic gain control, the digital signal is subjected to a filterbank 38 comprising a plurality of filters F1 . . . Fm (for example, band-pass filters) which are configured to divide the digital signal into m analysis channels 40, each containing a signal representative of a distinct frequency portion of the audio signal sensed by the microphone 20. For example, such frequency filtering may be implemented by applying a Discrete Fourier Transform to the audio signal and then distribute the resulting frequency bins across the analysis channels 40.

The signals within each analysis channel 40 are input into an envelope detector 42 in order to determine the amount of energy contained within each of the signals within the analysis channels 40 and to estimate the noise within each channel. After envelope detection the signals within the analysis channels 40 may be input into a noise reduction module 44, wherein the signals are treated in a manner so as to reduce noise in the signal in order to enhance, for example, the intelligibility of speech by the patient.

The optionally noise reduced signals are supplied to a mapping module 46 which serves to map the signals in the analysis channels 40 to the stimulation channels S1 . . . Sn. For example, signal levels of the noise reduced signals may be mapped to amplitude values used to define the electrical stimulation pulses that are applied to the patient 17 by the ICS 14 via M stimulation channels 52. For example, each of the m stimulation channels 52 may be associated to one of the stimulation contacts 19 or to a group of the stimulation contacts 19.

The sound processor 24 further comprises a stimulation strategy module 48 which serves to generate one or more stimulation parameters based on the noise reduced signals and in accordance with a certain stimulation strategy (which may be selected from a plurality of stimulation strategies). For example, stimulation strategy module 48 may generate stimulation parameters which direct the ICS 14 to generate and concurrently apply weighted stimulation current via a plurality 52 of the stimulation channels S1 . . . Sn in order to effectuate a current steering stimulation strategy. Additionally or alternatively the stimulation strategy module 48 may be configured to generate stimulation parameters which direct the ICS 14 to apply electrical stimulation via only a subset N of the stimulation channels 52 in order to effectuate an N-of-M stimulation strategy.

The sound processor 24 also comprises a multiplexer 50 which serves to serialize the stimulation parameters generated by the stimulation strategy module 48 so that they can be transmitted to the ICS 14 via the communication link 30, i.e. via the coil 28.

The sound processor 24 may operate in accordance with at least one control parameter which is set by a control unit 54. Such control parameters, which may be stored in a memory 56, may be the most comfortable listening current levels (MCL), also referred to as “M levels”, threshold current levels (also referred to as “T levels”), dynamic range parameters, channel acoustic gain parameters, front and back end dynamic range parameters, current steering parameters, amplitude values, pulse rate values, pulse width values, polarity values, the respective frequency range assigned to each electrode and/or filter characteristics. Examples of such auditory prosthesis devices, as described so far, can be found, for example, in WO 2011/032021 A1.

According to the example shown in FIG. 1, the stimulation system comprises a hearing aid 21 for additional acoustic stimulation of the patient's hearing which may be integrated within the Ci device 10 in order to stimulate the ipsilateral ear (thereby forming, together with the CI device 10, an EAS unit 31) or which may be provided separately at the contralateral ear in order to stimulate the contralateral ear (these variants are shown in dashed lines in FIG. 1). The hearing aid 21 comprises a microphone arrangement 29 for capturing audio signals from ambient sound (or may use the microphone 20 of the CI device), an audio signal processing unit 27 for processing the captured audio signals, and the loudspeaker 23 to which the processed audio signals are supplied.

FIG. 5 is a diagram showing various examples of mapping schemes, i.e. frequency allocation functions, which may be programmed by the fitting device 13 into the sound processor 24 to be used by the mapping module 46. The frequency allocation schemes of FIG. 5 show how the analysis channels, i.e. the frequencies of the input audio signal, are distributed onto the stimulation channels, i.e. the electrodes 18.

Generally, the pitch perception by the patient is determined by the frequency allocation function. An example of an initial mapping scheme/frequency allocation curve is shown at “A” in FIG. 5, with the lower cutoff frequency being indicated at fₘᵢₙ₁ and with the upper cutoff frequency being indicated at fₘₐₓ.

A more practical example of an initial frequency allocation curve A is shown in FIG. 6 which is a schematic representation of an example of a graphical user interface 100 implemented in the fitting device 13. According to the example of FIG. 6, the initial frequency allocation curve may be based on a logarithmic function, resulting in a straight line in the diagrams of FIGS. 5 to 8 having a logarithmic frequency axis. For example, the lower cutoff frequency fₘᵢₙ₁ may be 350 Hz, and the upper cutoff frequency fₘₐₓ₁ may be 5000 Hz. Such initial frequency allocation curve may be preprogrammed into the sound processor 24 at the manufacturer as a default setting, or it may be the result of a first fitting session.

For several reasons, it may be necessary to modify the present frequency allocation curve, for example, when acoustic stimulation is added to a system which was previously used for electric stimulation only. In such cases it may be desirable to increase the lower cutoff frequency of the electric stimulation in order to avoid an undesired frequency overlap with the acoustic stimulation. For example, it may be desirable to increase the lower cutoff frequency from 350 Hz to 850 Hz, as shown in the example of FIG. 6, where the modified lower cutoff frequency is indicated at fₘᵢₙ₂. The modified lower cutoff frequency may be selected by the user of the fitting device 13 (typically an audiologist) via the user interface 100 (in the examples of FIGS. 6 to 8 a slider labelled “EAS cross-over frequency”) is provided to this end.

Alternatively or in addition, the fitting device 13 may provide for a default setting of such modified lower cutoff frequency fₘᵢₙ₂.

For example, the modified lower cutoff frequency may be selected such that the upper cutoff frequency of the acoustic stimulation is not more than the modified lower cutoff frequency of the electric stimulation. Typically, the lower cutoff frequency of the acoustic stimulation is lower than the modified lower cutoff frequency of the electric stimulation, while the upper cutoff frequency of the acoustic stimulation may be equal to or higher than the modified lower cutoff frequency of the electric stimulation, but significantly lower than the upper cutoff frequency of the electric stimulation.

Usually, complete re-allocation of the analysis channels is not desired by the patient due to the fact that the patient's pitch perception is adapted to the frequency allocation function used so far. In order to avoid unpleasant hearing impressions by the patient as far as possible, the user of the fitting device 13 is enabled to select a matching frequency for the modified mapping scheme/frequency allocation curve. The selection of the mapping frequency has the effect that the allocation of frequencies above the matching frequency up to the upper cutoff frequency remains unchanged, whereas the frequencies below the matching frequency down to the modified lower cutoff frequency are re-allocated onto the stimulation channels according to the value of the modified lower cutoff frequency fₘᵢₙ₂.

Preferably, the same type of allocation function is used for re-allocating the frequencies below the matching frequency as is used for allocating the frequencies below the matching frequency in the initial mapping scheme (this means in the example of FIG. 6 that for the modified frequency allocation curve a logarithmic function is used also below the matching frequency, however, with a steeper slope as in the initial allocation curve and in the frequency range above the matching frequency). In the example of FIG. 6, the matching frequency of the modified allocation curve is indicated at f_{match2}, with a value of about 3000 Hz. The modified allocation curve is labelled “B” in FIGS. 5 and 6.

The fitting device 13 may provide some guidance to the user with regard to the selection of the matching frequency. For example, the fitting device 13 may suggest a default value which is determined as a function of the ratio of the modified lower cutoff frequency to the upper cutoff frequency. For example, the default value of the matching frequency may be selected such that it corresponds to the stimulation channel in the middle (corresponding to a 50% ratio, i.e. 50% of the relevant electrodes/electrode range) between the stimulation channel which corresponded in the initial mapping scheme to the modified lower cutoff frequency (channel #6 in the example of FIG. 6) and the stimulation channel corresponding to the upper cutoff frequency (which is not changed in the example of FIG. 6 and corresponds to channel #12); in the example of FIG. 6 such default value for an in this case foreseen 50% ratio would correspond to channel #11 (corresponding to a frequency of about 2500 Hz in the initial mapping curve). The ratio required to determine the default matching frequency can be fixed or configurable within the fitting device 13.

Alternatively or in addition, the fitting device 13 may propose a range from which the matching frequency may be selected. According to one example, the upper limit of this selectable range may be increased with increasing total use time of the cochlear implant device, allowing for a more pronounced re-allocation once the patient already has adapted to a preceding re-allocation.

In general, the selection of a modified mapping scheme may be repeated at a later point in time in order to allow for gradual adjustment of the mapping scheme so as to enable the patient to gradually adapt to changes in the frequency allocation. Such repeatedly selected modified mapping schemes may have different lower cutoff frequencies and/or different matching frequencies. In the example of FIG. 5 a modified frequency allocation curve C is shown, which has the same modified lower cutoff frequency fₘᵢₙ₂ but a higher matching frequency f_{match3} compared to the curve B.

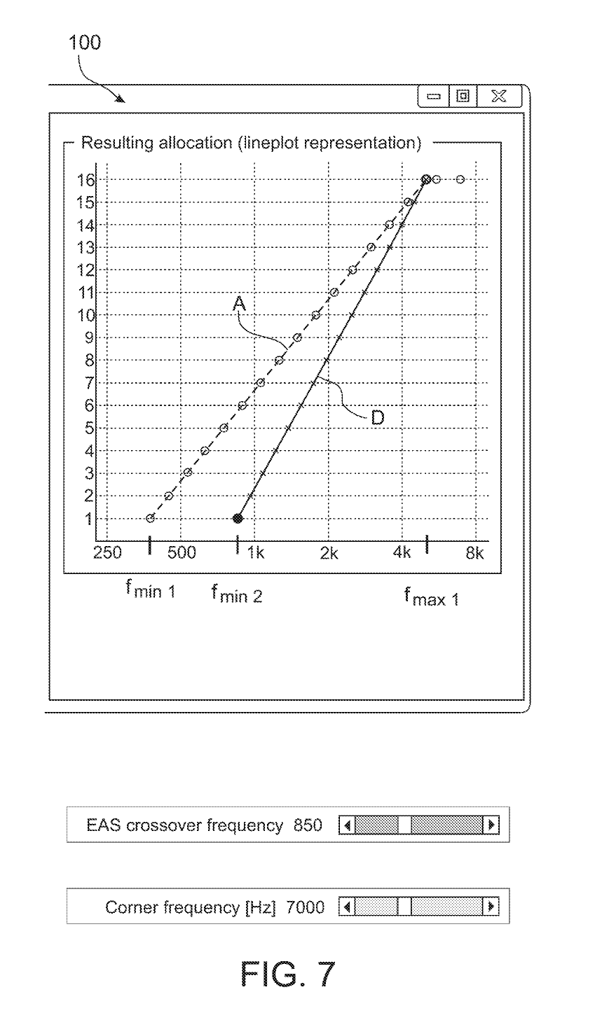

According to one example, the matching frequency may be increased even up to the initial upper cutoff frequency fₘₐₓ₁; examples of such modified allocation curves are shown at D in FIG. 5 and allocation curve D in FIG. 7 (in the example of FIG. 7, all analysis channels are reallocated according to a logarithmic function, resulting in a steeper slope in the logarithmic representation of FIG. 7).

The above discussed concept of a variable/selectable matching frequency may be applied also to cases in which there is no need to preserve an adaptation of the patient to a previous frequency allocation. Such cases may occur, for example, if a

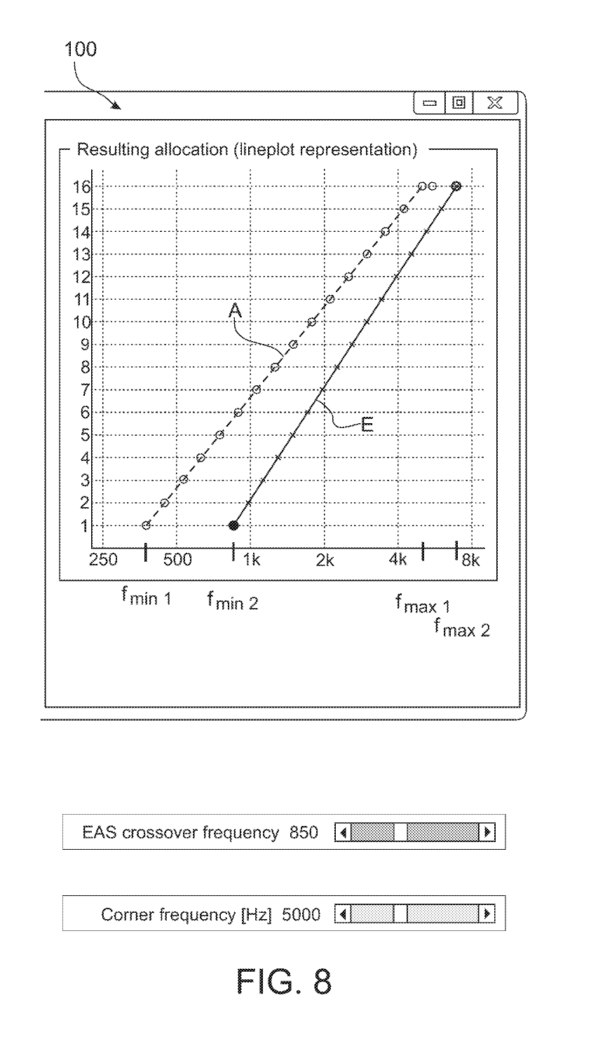

CI device is adapted for the first time to a new patient in order to provide for an individual setting for a the patient (in this case the modification of the mapping scheme would correspond to an adjustment/modification of a default frequency allocation function, such as curve A in FIGS. 6 to 8). In cases in which there is no need for an adaptation-preserving frequency allocation adjustment, the matching frequency may be selected to be higher than the initial upper cutoff frequency fₘₐₓ₁, such matching frequency is indicated at fₘₐₓ₂ in FIGS. 5 and 8, and the corresponding modified frequency allocation curve is indicated at “E” in FIGS. 5 and 8; the modified frequency allocation curves E also have a modified lower cutoff frequency fₘᵢₙ₂, as in the examples of the curves B, C and D in FIGS. 5 to 7.

In the example of FIG. 8, the resulting modified frequency allocation curve E is a straight line due to the logarithmic representation having, compared to the initial allocation curve A, a different slope and a different interception with the frequency axis.

According to one aspect of the invention, a default value of the modified upper cutoff frequency is selected as a function of the value of the modified lower cutoff frequency fₘᵢₙ₂ such that the bandwidth of each analysis channel is substantially the same as in the initial mapping scheme, so that the logarithmic bandwidth allocated to each stimulation channel/electrode is substantially independent from the actually selected modified lower cutoff frequency. For example, in case of a logarithmic allocation function, as in FIG. 8, all frequencies are shifted upwards by the same amount determined by the selected modified lower cutoff frequency fₘᵢₙ₂ (in the example of FIG. 8, the modified upper cutoff frequency fₘₐₓ₂ would be selected such that the distances between the channels would remain constant).

However, there may be a practical upper limit of the matching frequency, the bandwidth of the sound processor 24 or a maximum frequency relevant for speech perception (for example 8000 Hz), in case that the selected modified lower cutoff frequency is relatively high, such as 1600 Hz (in this case, for a logarithmic allocation function, the upper cutoff frequency would have to be increased from about 5000 Hz to 26000 Hz in order to keep the channel logarithmic bandwidth constant). This problem can also be mitigated by allowing a decreased logarithmic channel bandwidth as a function of the modified lower cutoff frequency, such that the practical upper limit of the matching frequency is just reached with the highest expected modified lower cutoff frequency.

Typically, the selected modified lower cutoff frequency will be higher than the default initial lower cutoff frequency.

1. A hearing assistance system comprising a cochlear implant device for neural stimulation of the cochlea of one of the patient's ears and a fitting device for adjusting the cochlear implant device; the system including means for providing an input audio signal;
the cochlear implant device comprising:
a sound processor for generating a neural stimulation signal from at least part of the input audio signal,
a cochlear implant stimulation arrangement comprising a plurality of stimulation channels for cochlear stimulation at various stimulation sites according to the neural stimulation signal, with each stimulation channel being attributed to a certain one of the stimulation sites,
the sound processor comprising a filterbank for dividing the input audio signal into a plurality of analysis channels and a mapping unit for allocating all analysis channels having a frequency between a lower cutoff frequency and an upper cutoff frequency to the stimulation channels according to an adjustable mapping scheme;
the fitting device comprising a user interface for selecting a value of a matching frequency, the fitting device being adapted to program the cochlear implant device in such a manner that the mapping scheme is changed from an initial mapping scheme having, as the lower cutoff frequency, an initial lower cutoff frequency and having, as the upper cutoff frequency, an initial upper cutoff frequency, to a modified mapping scheme having, as the lower cutoff frequency, a modified lower cutoff frequency which is higher than the initial lower cutoff frequency and having, as the upper cutoff frequency, the initial upper cutoff frequency,
wherein in the modified mapping scheme the allocation of the analysis channels having a frequency above the matching frequency and up to the initial upper cutoff frequency remains unchanged with regard to the initial mapping scheme, and wherein the analysis channels having a frequency between the modified lower cutoff frequency and the matching frequency are re-allocated by being distributed onto those stimulation channels to which, in the initial mapping scheme, the analysis channels having a frequency between the initial lower cutoff frequency and the matching frequency had been allocated, with the selectable matching frequency being lower than the initial upper cutoff frequency and higher than the modified lower cutoff frequency.

2. The system of claim 1, wherein the user interface is adapted for selection of the modified lower cutoff frequency by a user.

3. The system of claim 1, wherein the fitting device is adapted to provide for a default value of the modified lower cutoff frequency.

4. The system of claim 1, wherein in the initial mapping scheme the analysis channels are allocated to the stimulation channels according to a monotonous function, and wherein in the modified mapping scheme the analysis channels are allocated to the stimulation channels according to a monotonous function.

5. The system of claim 4, wherein in the initial mapping scheme the analysis channels are allocated to the stimulation channels according to a first logarithmic function.

6. The system of claim 5, wherein in the modified mapping scheme the analysis channels between the modified lower cutoff frequency and the matching frequency are allocated to the stimulations channels according to a second logarithmic function.

7. The system of claim 1, wherein the matching frequency is selectable within a given range from a lower limit matching frequency and an upper limit matching frequency.

8. The system claim 1, wherein the matching frequency is selectable to be set to a default value determined as a function of the ratio between the modified lower cutoff frequency and the initial upper cutoff frequency.

9. The system of claim 1, wherein the fitting device is adapted to enable the user to select a new modified mapping scheme having a different modified lower cutoff frequency and/or a different matching frequency.

10. The system of claim 9, wherein the fitting device is adapted to enable the user to select a new modified mapping scheme having a matching frequency higher than initial upper cutoff frequency, wherein all analysis channels are re-allocated to the stimulation channels according to a monotonous function.

11. The system of claim 10, wherein all analysis channels are re-allocated to the stimulation channels according to a logarithmic function.

12. The system of claim 1, wherein the fitting device is adapted to enable the user to select a new modified mapping scheme having the same modified lower cutoff frequency as a previous modified mapping scheme.

13. A hearing assistance system comprising a cochlear implant device for neural stimulation of the cochlea of one of the patient's ears and a fitting device for adjusting the cochlear implant device; the system including means for providing an input audio signal;
the cochlear implant device comprising:
a sound processor for generating a neural stimulation signal from at least part of the input audio signal,
a cochlear implant stimulation arrangement comprising a plurality of stimulation channels for cochlear stimulating at various stimulation sites according to the neural stimulation signal, with each stimulation channel being attributed to a certain one of the stimulation sites,
the sound processor comprising a filterbank for dividing the input audio signal into a plurality of analysis channels and a mapping unit for allocating all analysis channels having a frequency between a lower cutoff frequency and an upper cutoff frequency to the stimulation channels according to an adjustable mapping scheme using a monotonous function;
the fitting device comprising a user interface for selecting a value of a modified lower cutoff frequency and being adapted to program the cochlear implant device in such a manner that the mapping scheme is changed from an initial mapping scheme having, as the lower cutoff frequency, an initial lower cutoff frequency and having, as the upper cutoff frequency, an initial upper cutoff frequency, to a modified mapping scheme having, as the lower cutoff frequency, the selected value of the modified lower cutoff frequency and having, as the upper cutoff frequency, a matching frequency,
wherein in the modified mapping scheme the analysis channels are re-allocated to the stimulation channels by being distributed onto the stimulation channels, and wherein the matching frequency is determined as a function of the selected value of the modified lower cutoff frequency such that the bandwidth of each analysis channel is substantially the same as in the initial mapping scheme.

14. The system of claim 13, wherein the matching frequency is restricted to values below an upper limit.

15. The system of claim 13, wherein the selectable values of the modified lower cutoff frequency are higher than the initial lower cutoff frequency.

16. The system of claim 13, wherein in the initial mapping scheme the analysis channels are allocated to the stimulation channels according to a first logarithmic function.

17. The system of claim 16, wherein in the modified mapping scheme the analysis channels are allocated to the stimulation channels according to a second logarithmic function.

18. The system of claim 13, wherein the same type of mapping function is applied in initial mapping scheme and modified mapping scheme.

19. The system of claim 13, wherein the system further comprises a hearing aid for acoustic stimulation of the patient's hearing at the same one or the other one of the patient's ears, the hearing aid comprising an audio signal processing unit for processing at least part of the input audio signal, and an acoustic output transducer for stimulating the patient's hearing according to the processed audio signals, wherein the processed audio signals have a lower cutoff frequency lower than the modified lower cutoff frequency of the modified mapping scheme and an upper cutoff frequency equal to or higher than the modified lower cutoff frequency of the modified mapping scheme but lower than the upper cutoff frequency of the modified mapping scheme.

20. The system of claim 19, wherein the upper cut-off frequency of the processed audio signals is greater than the modified lower cut-off frequency of the modified mapping scheme.

21. The system of claim 13, wherein each stimulation channel is associated to a different one of the electrodes of an implantable cochlear electrode array.

22. A method of adapting a hearing assistance system comprising a cochlear implant device for neural stimulation of the cochlea of one of the patient's ears; the system including means for providing an input audio signal; and the cochlear implant device comprising a sound processor for generating a neural stimulation signal from at least part of the input audio signal, a cochlear implant stimulation arrangement comprising a plurality of stimulation channels for cochlear stimulating at various stimulation sites according to the neural stimulation signal, with each stimulation channel being attributed to a certain one of the stimulation sites, the sound processor comprising a filterbank for dividing the input audio signal into a plurality of analysis channels and a mapping unit for allocating all analysis channels having a frequency between a lower cutoff frequency and an upper cutoff frequency to the stimulation channels according to an adjustable mapping scheme, the method comprising:
selecting, by a user via a user interface of the fitting device, a value of a matching frequency,
programming, by the fitting device, the cochlear implant device in such a manner that the mapping scheme is changed from an initial mapping scheme having, as the lower cutoff frequency, an initial lower cutoff frequency and having, as the upper cutoff frequency, an initial upper cutoff frequency, to a modified mapping scheme having, as the lower cutoff frequency, a modified lower cutoff frequency which is higher than the initial lower cutoff frequency and having, as the upper cutoff frequency, the initial upper cutoff frequency,
wherein in the modified mapping scheme the mapping of the analysis channels having a frequency above the matching frequency and up to the initial upper cutoff frequency remains unchanged with regard to the initial mapping scheme, and wherein the analysis channels having a frequency between the modified lower cutoff frequency and the matching frequency are re-allocated by being distributed onto those stimulation channels to which, in the initial mapping scheme, the analysis channels having a frequency between the initial lower cutoff frequency and the value of the matching frequency had been allocated, with the selectable matching frequency being lower than the initial upper cutoff frequency and higher than the modified lower cutoff frequency.

23. A method of adapting a hearing assistance system comprising a cochlear implant device for neural stimulation of the cochlea of one of the patient's ears; the system including means for providing an input audio signal; and the cochlear implant device comprising a sound processor for generating a neural stimulation signal from at least part of the input audio signal, a cochlear implant stimulation arrangement comprising a plurality of stimulation channels for cochlear stimulating at various stimulation sites according to the neural stimulation signal, with each stimulation channel being attributed to a certain one of the stimulation sites, the sound processor comprising a filterbank for dividing the input audio signal into a plurality of analysis channels and a mapping unit for allocating all analysis channels having a frequency between a lower cutoff frequency and an upper cutoff frequency to the stimulation channels according to an adjustable mapping scheme using a monotonous function, the method comprising:
selecting, by a user via a user interface of the fitting device, a value of a modified lower cutoff frequency,
programming, by the fitting device, the cochlear implant device in such a manner that the mapping scheme is changed from an initial mapping scheme having, as the lower cutoff frequency, an initial lower cutoff frequency and having, as the upper cutoff frequency, an initial upper cutoff frequency, to a modified mapping scheme having, as the lower cutoff frequency, a the selected value of the modified lower cutoff frequency which is higher than the initial lower cutoff frequency and having, as the upper cutoff frequency, a matching frequency,
wherein in the modified mapping scheme the analysis channels are re-allocated to the stimulation channels by being distributed onto the stimulation channels, and wherein the matching frequency is determined as a function of the selected value of the modified lower cutoff frequency such that the bandwidth of each analysis channel is substantially the same as in the initial mapping scheme.
